(19) United States
(12) Patent Application Publication
(10) Pub. No.: US 2018/0055373 A1
(43) Pub. Date: Mar. 1, 2018

(54) MONITORING DEVICE TO IDENTIFY CANDIDATES FOR AUTONOMIC NEUROMODULATION THERAPY

(71) Applicant: BIOTRONIK SE & Co. KG., Berlin (DE)

(72) Inventors: Lauren KRAITER, Tigard, OR (US); Dirk MUESSIG, West Linn, OR (US); Larry STOTTS, Tigard, OR (US)

(73) Assignee: BIOTRONIK SE & Co. KG., Berlin (DE)

(21) Appl. No.: 15/251,508

(22) Filed: Aug. 30, 2016

(51) Int. Cl. A61B 5/0205; A61B 5/00; A61B 5/0456; A61B 5/11

(52) U.S. Cl. CPC A61B 5/0205; A61B 5/6833; A61B 5/0456; A61B 5/02438; A61B 5/0022; A61B 5/1118; A61B 5/742

(57) ABSTRACT

A monitoring system and method for measuring and analyzing physiological signals to assess whether neuromodulation therapy would be successful for a patient. The system includes an external patch device that can be adhered to the chest of the patient in order to measure physiological signals, including heart signals via ECG and respiration via impedance measurement. An acceleration sensor is also provided in order to derive patient posture and activity levels that can be correlated to the measured data.

### FIELD OF THE INVENTION

The present invention relates to a monitoring device for observing an autonomic balance of a patient and assessing whether neuromodulation therapy would be appropriate for the patient.

### BACKGROUND OF THE INVENTION

The field of wearable health monitors is a recently created field of development due to the reduced size of memory, batteries and processors. These health monitors typically monitor heartbeat, the number of footsteps taken, body temperature, or other directly measurable physiological signals. Even the direct measurement of these physiological signals requires advanced processing to stabilize the signal and filter out the noise. Thus, some wearable monitors are merely sensors which relay the raw signal to a more powerful computer or medical device.

These wearable devices or sensors are often simply wireless versions of hardwired sensors used in hospitals in the past. The sensors transmit the sensed raw signals to a mobile phone, smartwatch or desktop computer for analysis. After analysis, the output remains only a basic physiological signal that would require interpretation by a medical or fitness professional. Furthermore, the combination of various signals or the usable baseline is rarely calculated, making these sensors medically primitive.

For instance, Toth, et al. (US 2015/0335288) discloses a number of configurations and designs for wearable medical sensors including clothing designs for implantation. The sensor device disclosed in Toth includes dozens of micro-sensors and a few macro-sensors which are collected by a centralized analog-to-digital converter and then passed to a processor for analysis. These micro-sensors can include minimally-invasive sensors or non-invasive monitors that are embedded in a pad and are applied directly to the skin.

These sensors can include an electrophysiologic sensor, a temperature sensor, a thermal gradient sensor, a barometer, an altimeter, an accelerometer, a gyroscope, a humidity sensor, a magnetometer, an inclinometer, an oximeter, a colorimetric monitor, a sweat analyte sensor, a galvanic skin response sensor, an interfacial pressure sensor, a flow sensor, a stretch sensor, or a microphone. Thus, many physiological and environmental variables can be collected, providing data to assist with diagnosis. A device containing all these sensors, though, would be exceedingly expensive and not entirely useful to a regular user with no medical experience.

One such device for detecting heart rate is that described in “ECG Patch Monitors for Assessment of cardiac Rhythm Abnormalities” by S. Suave Lobodzinski. This device is a patch monitor that includes a processor for ECG signal acquisition, amplification and filtering, a 12-bit Analog to Digital Converter (ADC) that converts the analog ECG signal into a digital format, and a custom Digital Signal Processor (DSP) responsible for various ECG processing tasks such as signal filtering, feature extraction, waveform analysis and motion artifact removal. The artifact removal is aided by an accelerometer, which provides time-dependent data on the patient's movements. The device of Lobodzinski also includes a BLUETOOTH transmitter for transmitting the filtered and extracted ECG signal.

Since the amount of correction required can be significant and can depend on several environmental variables, the simple extraction of the physiological signals from the sensors above can require Fast Fourier Transforms (FFT), Hilbert-Huang transforms, Hanning, Hamming, and Kaiser windows, Kalman filters, Bayesian filters or other adaptive filters. The application of these algorithms has been the forefront of the medical device industry. Though these algorithms can accurately isolate a signal, the resulting physiological signals have to be further adapted to each person's baseline and compared with demographic averages.

Thus, many wearable medical devices have been developed to sense physiological signals accurately, but do not aid in the interpretation of these signals. Specifically, heart rhythm and variability can be analyzed for several diseases but without the context of other signals, past signals and environmental context, the signal alone is ill-suited for diagnosis. Furthermore, a wearable device targeting a certain disease should have all the necessary sensors affecting the diagnosis and can have disease-specific requirements for detection.

These specialized medical devices to aid in diagnosis using long-term data collection have yet to be developed for most diseases. Chronic diseases and especially age-related diseases must be monitored in the long-term and in context in order to accurately determine seriousness and progress of the disease.

Numerous conditions are associated with autonomic imbalance, such as hypertension, heart failure, ventricular arrhythmia risk, sleep apnea, diabetes, and others. Autonomic neuromodulation therapies, such as vagus nerve stimulation, spinal cord stimulation, and baroreceptor stimulation, seek to address these conditions via stimulation of the nervous system to restore autonomic balance. However, preliminary clinical studies reveal that, as with many interventions, some patients display a significant benefit and are considered to be responders to the therapy, while other patients do not show a significant favorable change as a result of therapy.

Additionally, recent clinical studies have failed to show a statistically significant response to neuromodulation therapy, likely because inadequate selection criteria were used for identifying candidate patients. Despite the recognition that there are responders and non-responders to autonomic neuromodulation therapy, no tools exist for discriminating between patients to identify likely responders prior to referral for device implant.

### SUMMARY OF THE INVENTION

It is therefore an object of the present invention to provide a system that measures sensor signals from a variety of sources and evaluates these physiological signals in an ongoing basis to assess whether neuromodulation therapy would be successful for the patient. The monitor can use an external patch device that can be adhered to the chest of the patient in order to measure physiological signals, including heart signals via ECG and respiration via impedance measurement. An acceleration sensor is also provided in order to derive patient posture and activity level that can be correlated to the measured data. The device may perform filtering and processing of the acquired patient data.

For example, the data can be sent wirelessly to an external unit such as a handheld device for the physician or patient and/or to a remote service center. Once the data has been collected, a diagnostic process analyzes the data to determine the autonomic balance of the patient. Specifically, the heart rate data and respiratory data are examined and controlled using the accelerometer, with the heart rate being compared against expected thresholds.

The diagnostic process evaluates heart rate at rest (HRR) in connection with respiration. The diagnostic device detects and isolates the peaks in respiration and determines the maximum and minimum heart rate within a time window according to the respiration peaks. It then determines the difference between the minimum and maximum heart rate according to the respiration peaks. Also, the diagnostic device determines the average difference using a series of maximum and minimum differences, which is then quantified as the heart rate variability (HRV) specifically associated with respiration (HRVr). Other calculations of heart rate variability (HRV) may be used alternatively or in conjunction with this calculation.

The diagnostic method for evaluating the suitability for neuromodulation therapy is a combination of one or more cardiac variables with one or more threshold values. The cardiac variables are evaluated and compared to defined threshold values in a step-wise fashion, and the results are input into a decision tree for determining whether a patient is a good candidate for neuromodulation therapy. In one exemplary embodiment, the evaluation of suitability for neuromodulation therapy includes a stepwise approach of evaluating heart rate at rest, atropine response, and heart rate variability. In one embodiment, the comparison is performed by a processor or a processing unit.

In this embodiment, the evaluation of heart rate at rest (HRR) may include two threshold heart rates, a and b, wherein a<b, and if HRR<a, then the natural vagal tone of the patient is acceptable and the patient is not a candidate for neuromodulation therapy. If the diagnostic device determines that a<HRR<b, the device suggests testing the patient with an administration of atropine. The result of the atropine test contributes to determining whether the treatment for the patient is suitable or not. In the case of a blunted heart rate response to atropine in combination with a<HRR,b, or in the case that b<HRR, then HRV is checked to determine whether a threshold d is crossed. If the threshold d is crossed, then the variability is too high and the patient is also not suited for neuromodulation therapy. Otherwise, if HRV<d in combination with risk factors assessed by the previous tests, the patient is suited for neuromodulation therapy.

An advantage of this diagnostic process is an improved risk-benefit ratio for patients so that those patients who are more likely to respond to neurostimulation therapy will be selected for the implant. The diagnostic process also allows for pre-screening patients for a clinical study. This increases the likelihood of a successful clinical study and increases likelihood of approval of new therapies as well as post-market studies for additional therapy claims. By automating the pre-screening process, a larger number of patients are likely to be considered for the treatment implant.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitative of the present invention, and wherein:

FIG. 1 is an overview of the system communication network;

FIG. 2 is an diagram of the initial processing of the system;

FIG. 3 is an illustration of the wearable device;

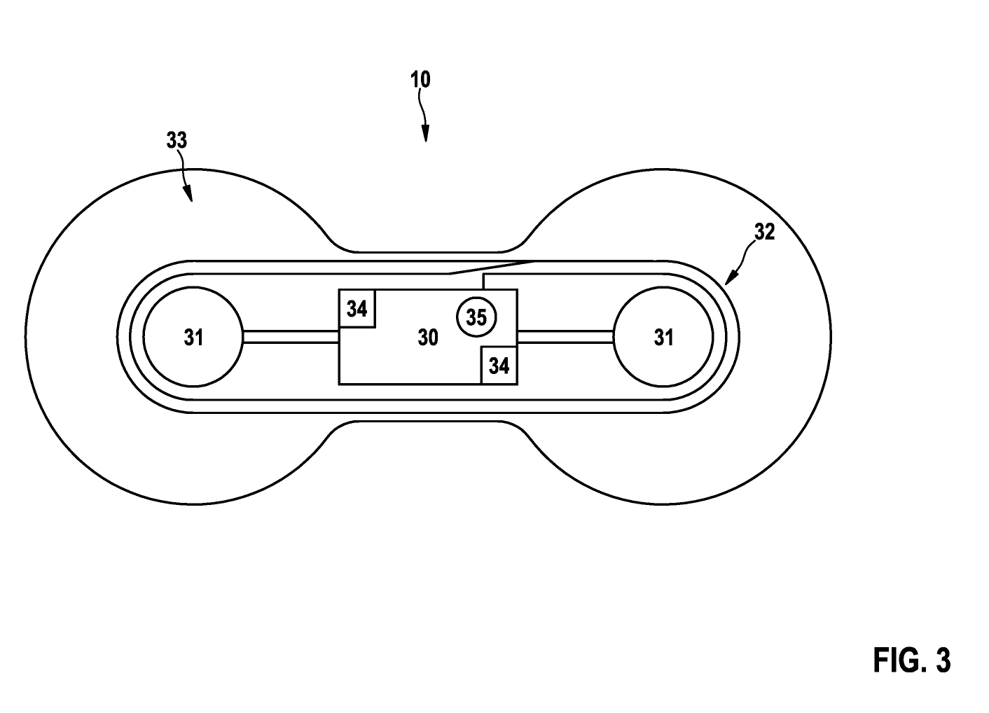

FIG. 4 is a diagram of the long-term data collection and processing for the heart rate at rest;

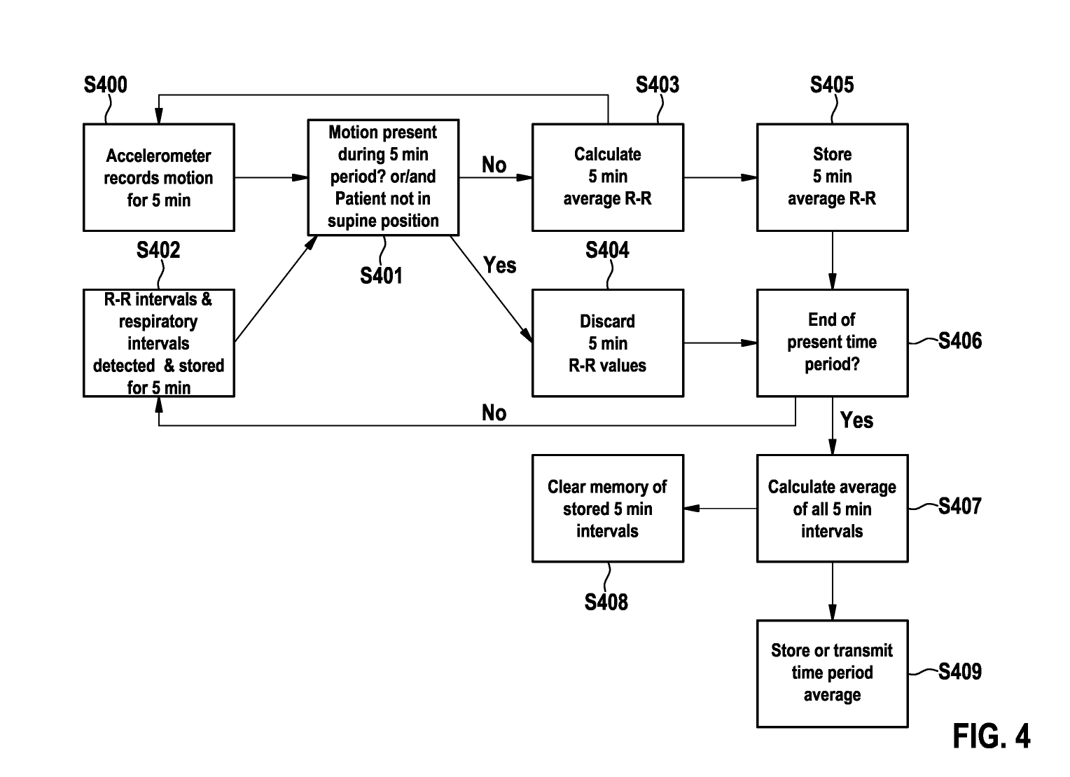

FIG. 5 is an annotated heart rate and respiratory record according to an embodiment of the system;

FIG. 6 is a graph of the normal response to atropine dosage levels;

FIG. 7 is an annotated graph of an atropine test; and

FIG. 8 is an algorithm for determining candidates of neuromodulation therapy.

### DETAILED DESCRIPTION OF THE DRAWINGS

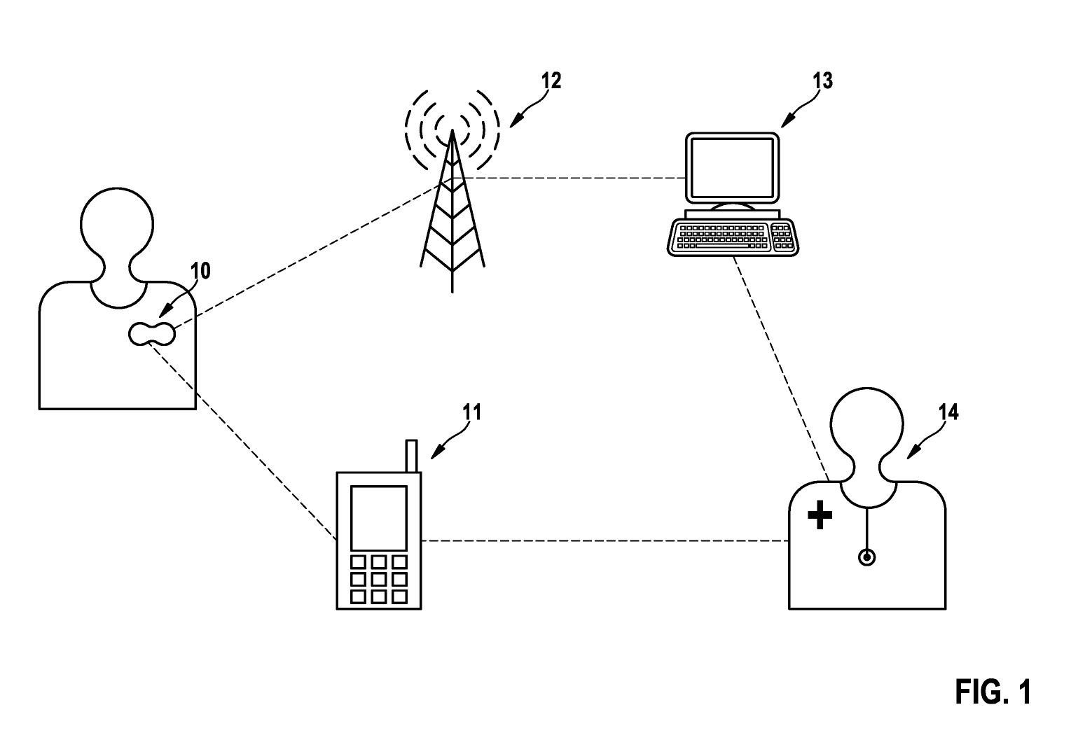

The external monitoring system that provides an assessment of intrinsic autonomic imbalance is shown in FIG. 1. The monitoring system includes an external wearable device 10 and either a physician's mobile device 11 or, for example, a mode of transmission 12 to an internet-based service center 13. The wearable device 10 stores information until it is either interrogated by the clinician's mobile device 11, and/or until it can be transmitted to the internet service center 13. In the case of transmission to an internet service center 13, the wearable device 10 may contain cellular or wireless internet capability that allows it to transmit directly to the internet service center 13. In either case, the physician 14 is then able to view the compiled and processed results.

The wearable device 10 may also communicate via radio frequency with a mobile device 11 used by the patient. The patient's mobile device 11 then has either a cellular or wireless or wired internet connection for sending the information to the internet service center 13. The patient may wish to view the daily changes or view the treatment response even if they are unable to interpret the signals.

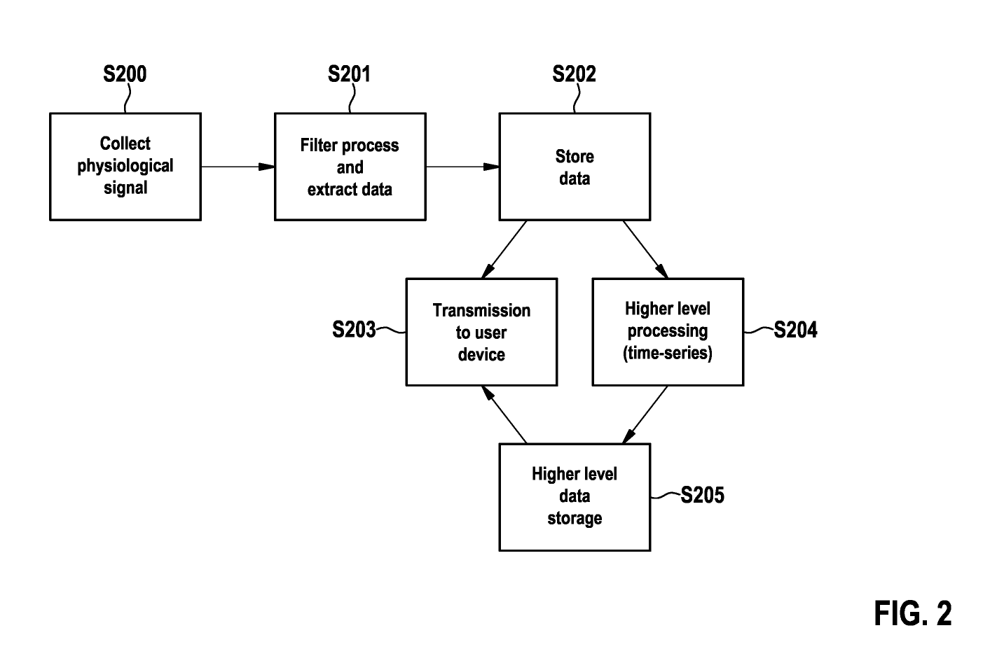

The overview of the process performed by the wearable device 10 is shown in FIG. 2. The wearable device 10 first collects the physiological signal S200, then filters, processes and extracts the signal data in real-time S201, then stores the filtered/processed data S202. This locally stored data is then transmitted to the mobile device 11 or service center 13 in step S203, and/or the stored data is processed and filtered further in S204 by utilizing a longer time series, for example. This additionally processed data is then also stored S205 and transmitted to the mobile device 11 or service center 13 as in step S203.

According to an exemplary embodiment, the wearable device 10 includes at least two electrodes 31 enclosed in a water-resistant, self-adhesive patch 33 designed to be worn by the patient for several days to weeks. The electrodes 31 sense relevant electrical physiological signals such as chest electrocardiogram (ECG) and impedance signals that can indicate respiration. Additionally, the wearable device 10 may include an accelerometer 34 for detecting patient activity levels and/or postural information. It may include a trigger button or buttons 35 through which the patient or physician can indicate the start of an event. The physiological signals from the electrodes 31 and the accelerometer 34 are received by a processor in integrated circuit 30. The integrated circuit 30 does preliminary processing as shown in FIG. 2 and also stores processed and unprocessed data between processing and transmission periods.

Finally, the wearable device 10 includes some components for communication, for example, wireless internet communication directly to an internet service center, cellular communication to an internet service center, radiofrequency communication to a patient device (such as a monitor in the house) or clinician device (such as an in-office programmer), and/or near-field induction communication to a patient device or clinician device. The communication is performed over the embedded antenna 32 of the wearable device 10 and controlled by a transceiver in the integrated circuit 30, where the integrated circuit is, for example, a flexible printed circuit board.

In order to evaluate intrinsic autonomic tone, the external monitoring system calculates and stores trends for one or more of the following parameters: average heart rate, resting heart rate, short-term heart rate variability, heart rate variability in relation to respiration, heart rate variability at rest, premature ventricular contraction (PVC) count, and the heart rate response to specific challenges.

Each of these parameters may be calculated from one or more physiologic signals that are collected by the wearable device 10. In one embodiment of the system, the processing and calculation of the parameters occurs within the hardware and software of the wearable component, and the calculated values are then stored for access via a clinician's mobile device or for transmission to an internet service center. In an alternative embodiment, the wearable component stores only raw values of physiologic signals, such as snapshots of the ECG or impedance trends, which are measured between electrodes via delivery of low-level current pulses delivered in a series of pulse per second. In this embodiment, the raw signals are acquired via the clinician's device or via the internet service center, after which the parameters of interest are derived. In a third intermediate embodiment, some of the processing may be performed within the wearable component, with additional processing performed by the clinician's device or internet service center.

Heart rate is known to be a function of both parasympathetic and sympathetic influences, and thus is a potential physiological parameter used by the external monitoring system for evaluating likelihood of response to autonomic neuromodulation. In one embodiment, this system uses the ECG signal to derive heart rate by detecting the occurrence of ventricular R-waves and calculating the interval between them (R-R intervals), where R is a point corresponding to the peak of the QRS complex of the ECG wave. The system stores heart rate values in order to calculate the average heart rate over a preset time period, for example, a 24 hour period. Furthermore, heart rate during times of rest can be a useful indication of intrinsic parasympathetic tone because sympathetic tone is withdrawn in the absence of exercise.

Therefore, alternatively or in addition to overall average heart rate, the system can use heart rate data along with data from the accelerometer to calculate a heart rate at rest or a nighttime heart rate S400. In one embodiment for calculating heart rate at rest, the system first evaluates if motion is present on the accelerometer S401, and if no motion is present, it then stores the heart rate values to use in calculating an average. In the case of nighttime heart rate, the intention is to calculate a heart rate average that is only representative of when the patient is sleeping.

According to an exemplary embodiment for calculating night time heart rate, the system first evaluates if the patient is in a supine position S401 according to three-dimensional orientation data from the accelerometer 34. If the patient is supine, the system evaluates if the patient is also motionless S401 according to the accelerometer. If both conditions are met, the system then calculates S403 and saves the average of the past interval of recorded heart rate values S405 for use in calculating the nighttime heart rate average. If one or both of the conditions fail then the heart rate values for the interval are discarded S404.

In an embodiment, the system stores R-R intervals and respiration intervals continuously as long as the requirements are met, and then after a preset time period (e.g. 24 hours) S406, the system calculates the average of all saved values S407. Alternatively, the system may store averages over smaller time intervals (e.g. 5 minutes) S405 during which the criteria are met, then after a preset period of time S406, average together all of the smaller interval averages into a final average. This final average for the entire day or for the nighttime is then stored or transmitted S409 and the memory storing the smaller interval averages or all the interval data is cleared.

The system also automatically restarts recording the accelerometer, heart rate and impedance from the electrodes 31 and accelerometers 34 after the end of each smaller time interval. Furthermore, if the preset period has not been reached, the system continues recording physiological signals into local memory. Alternatively, the system could generate a running average that is reset and output every 5 minutes or after 24 hours.

Heart rate variability (HRV), particularly the high frequency component associated with respiration, is known to be vagally mediated. Therefore, HRV is another potential physiological parameter that should be recorded. According to one embodiment, the HRV calculation used by the system is the SDNN index, in which the mean of the 5-minute standard deviations of the R-wave intervals is calculated over 24 hours. The system may also incorporate an ability to discriminate between normal R-waves (originating from atrial conduction) and PVCs, in order to include only normal R-waves into the calculation of HRV.

Likewise, HRV at rest may be a parameter of interest. Like the heart rate at rest described above, the HRV at rest is acquired by the system first evaluating if motion is present on the accelerometer, and if no motion is present, it then stores the HRV values for use in averaging a HRV at rest value. Alternatively or in addition to HRV based on R-R intervals alone, the system may also monitor breathing rate respiration according to thoracic impedance fluctuations in order to assess the variations in heart rate that are specifically associated with respiration.

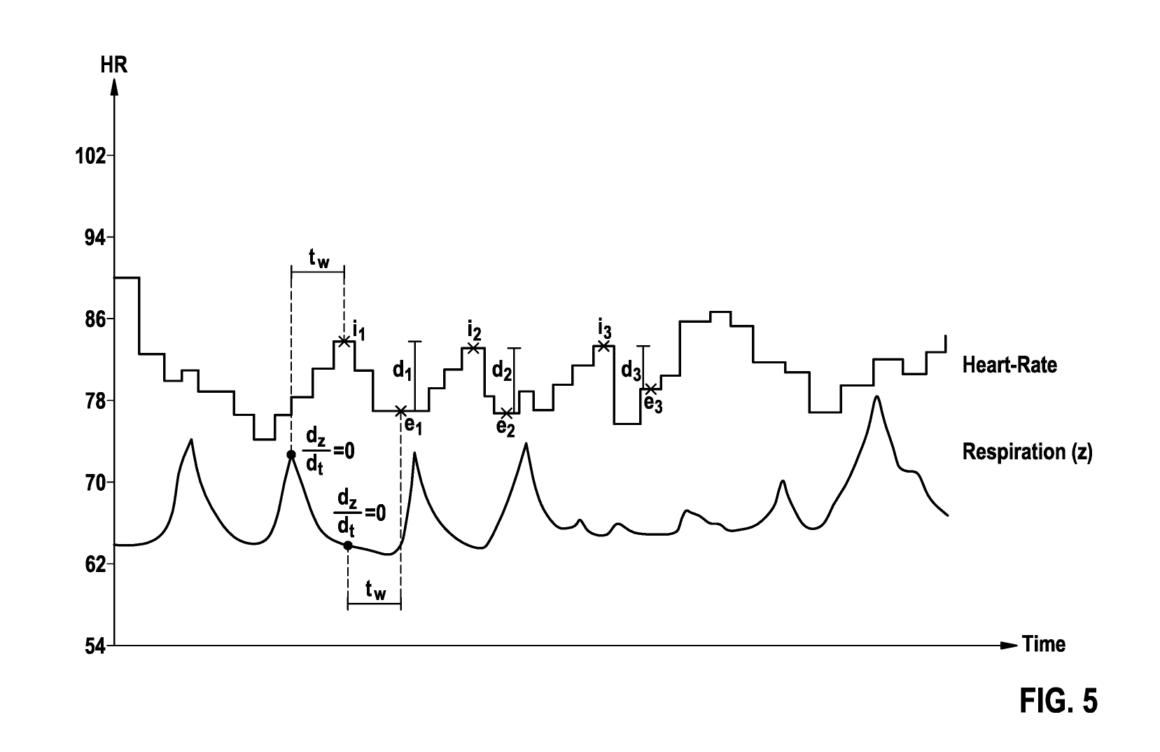

An illustration of HRV assessment with respiration is shown in FIG. 5 in graph format. Thoracic impedance measurements are acquired at a high sampling rate (multiple times per second) and saved in a buffer. The impedance signal (z) is analyzed to identify points where the derivative is equal to zero (dz/dt=0) in order to identify times at which the peaks of inspirations and expirations occurred. The heart rate on a beat-to-beat basis is also saved in a memory buffer during the same period.

For each peak of inspiration that is found, the algorithm searches for a peak heart rate within a time window (tw) and saves that heart rate value as iₙ (e.g. i₁, i₂, i₃). For each expiration that is found, the algorithm searches for a local minimum in the heart rate within time window tw following the expiration peak, and saves that heart rate value as eₙ. For each pair of respiration cycle heart rates, iₙ and eₙ, the algorithm calculates the difference dₙ between the values. Then, a series of differences (d₁, dₙ) are averaged to find the mean difference in heart rate between inspiration and expiration.

Premature ventricular contractions (PVCs) and other ventricular arrhythmias are known to be suppressed by vagal activity. Thus, the external monitoring system may also monitor the occurrence of PVCs to evaluate intrinsic autonomic influences. In order to distinguish PVCs from normal R-waves (originating from atrial conduction), the system may look for a deviation from the average R-R interval that exceeds a certain percentage change, or it may use more advanced forms of PVC detection such as morphology discrimination.

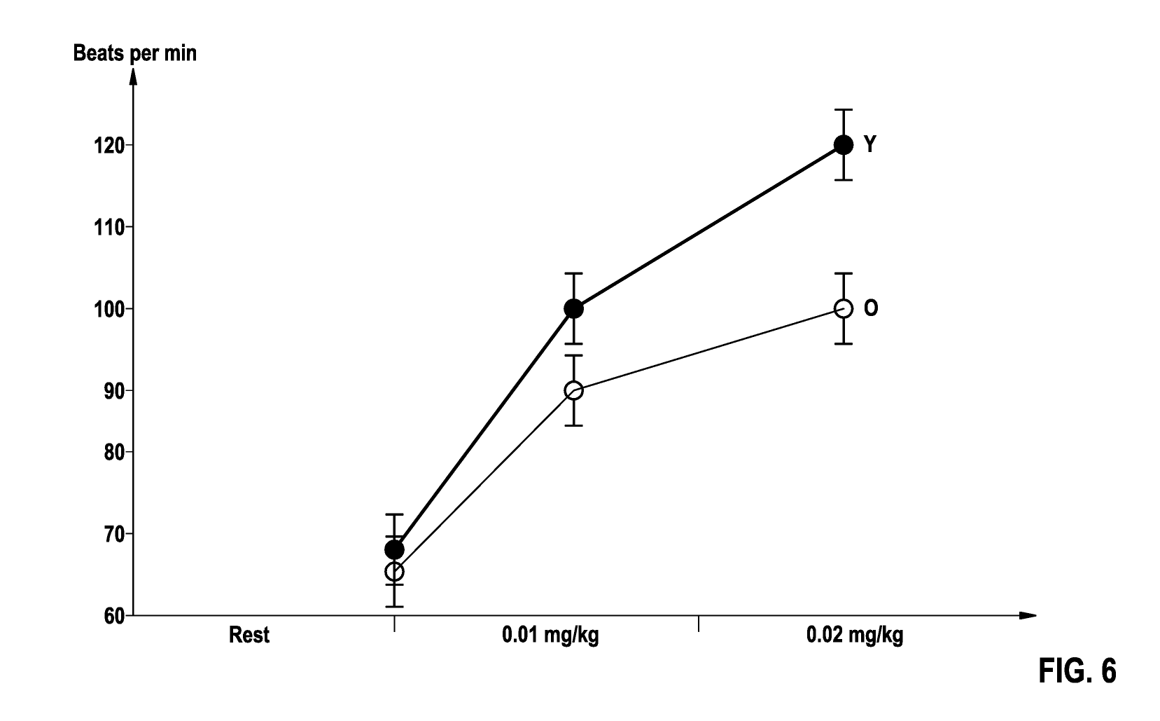

Finally, the external monitoring system may include monitoring of physiological response to special clinical test scenarios in order to evaluate intrinsic autonomic tone. For instance, the magnitude of average heart rate change in response to atropine administration is considered a gold standard for evaluating cardiac intrinsic vagal tone. As shown in FIG. 6, administration of atropine (0.01 mg/kg and 0.02 mg/kg) causes a marked heart rate increase in healthy individuals. Specifically, the curve for young adults is labeled “Y” and the curve for elderly adults is labeled “0”.

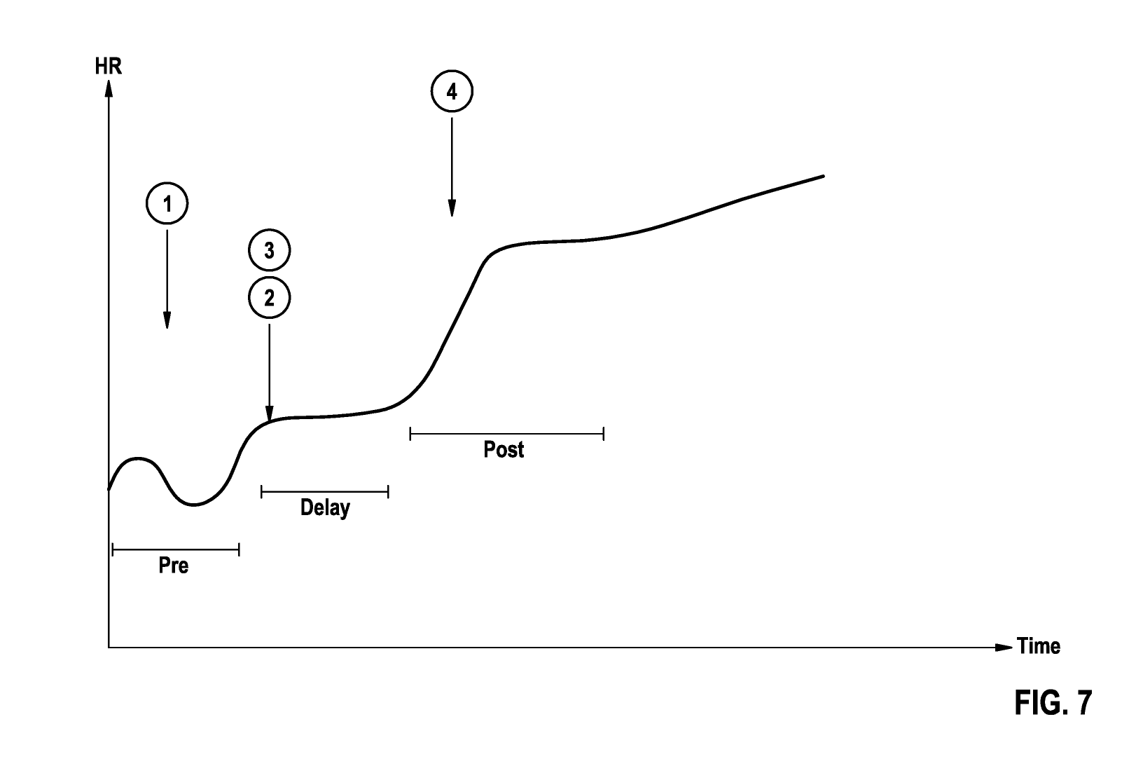

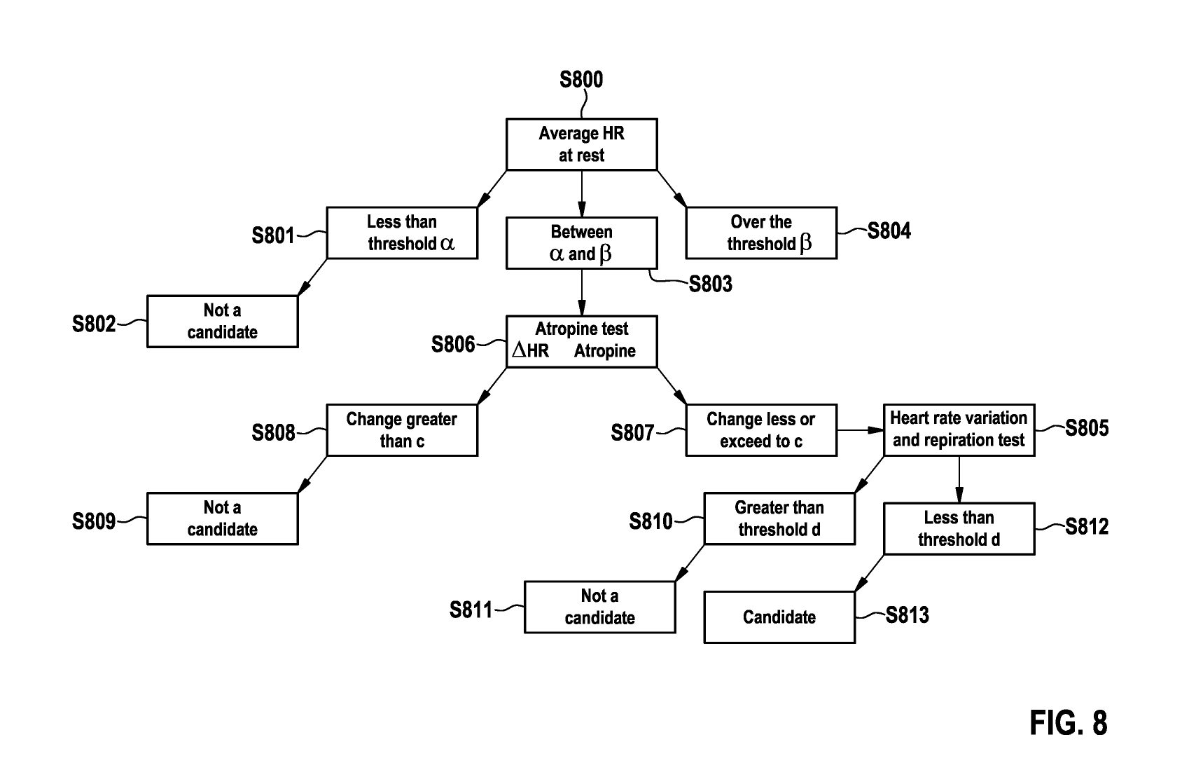

In individuals with impaired intrinsic vagal tone, the heart rate change in response to atropine is blunted. Based on these known physiological factors, the external monitoring device can perform a method to test for a heart rate response to atropine as shown in FIGS. 7 and 8. The graph in FIG. 7 illustrates a typical response to atropine as measured by the device with 1 designating the normal period before the dosage was administered. At 2 the dose is administered and a button 35 on the wearable device 10 is pressed at 3 to indicate that the test has begun. The device continues to monitor and record heart rate throughout the process as disclosed above.

As can be seen, the atropine dosage typically increases the heart rate significantly to a peak at 4. The three stages are also shown in FIG. 7 as the pre-test, the delay and then the post-period with the response. The system logs beat-to-beat heart rate data in a memory buffer in the pre-test time. Then, the clinician inputs the start of the atropine test just prior to injection of the atropine bolus, for example, through pressing a button for a designated time or number of presses on the wearable device 10. The system logs the time at which the test is started. The system continues to store heart rate data to the memory buffer for a delay period and a post-test period. The system finds HRpre, the average heart rate during the pre-test period, and HRₚₒₛₜ, the average heart rate during the post-test period. The ΔHRₐₜᵣₒₚᵢₙₑ is calculated as the difference HRpost−HRpre.

Other examples of specialized tests which may be incorporated in a similar fashion include: measuring the heart rate recovery change following an exercise period; heart rate response to tilt testing, heart rate response to a Valsalva maneuver, and heart rate response to phenylephrine infusion. For all of the physiological parameters collected by the system, the results could be displayed as summary trends for the physician to interpret. In an exemplary embodiment, or the system itself could process the results of multiple physiological parameter calculations to determine a recommendation of whether the patient is a candidate (e.g. likely to be a responder) for autonomic neuromodulation.

The process for analyzing the test as performed by the external monitoring device is shown in FIG. 8. In this embodiment, three different physiologic parameters are used. First, the averaged heart rate at rest is assessed S800 and compared to two thresholds (α and β). If the heart rate at rest is less than a S801, the patient has good vagal tone and is not a candidate for therapy S802. If the heart rate at rest is greater than a but less than β S803, an atropine test is required for further characterization of the resting heart rate.

In the case that the response to atropine is blunted (less than a threshold c) S807 and/or the heart rate at rest exceeds β S804, additional evaluation of HRV with respiration is performed S805 as described in FIG. 5. However, if the change in heart rate after the atropine dosage is greater than threshold c S808, then the patient is not a good candidate S809.

Finally, if HRV with respiration is greater than d S810, the patient does not have clear autonomic impairment and is not a good candidate S811; however, if HRV with respiration is less than d S812, there is clear evidence of vagal impairment and the patient is a good candidate S813 for neuromodulation therapy. For this system, some exemplary cutoff variables are shown in Table 1 below:

TABLE 1

| Variable | Threshold | Example Value |
|---|---|---|
| HR at rest lower threshold | α | 60 bpm |
| HR at rest upper threshold | β | 75 bpm |
| HR response to atropine | c | 30 bpm |
| HRV with respiration | d | 6 bpm |

The auto-screening of the candidates for neuromodulation therapy allows the physician to select the best possible patients for the response study without direct supervision. After some time at home or living in normal circumstances, the patient data collected can already rule out some candidates. The remaining candidates are then subjected to atropine tests. This reduces the upfront costs of the screening. The system also allows for automation of the atropine test.

The system sequences described above are exemplary and can be modified or combined. The recording intervals and the averaging period can be varied for different observation parameters. For instance, determining the nighttime heart rate at rest would not require a full 24 hours to be averaged. Likewise, the example thresholds listed above can change for young and old candidates or other patient variations.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A method for assessing a suitability of neuromodulation therapy for a patient, the method comprising:
detecting and storing, via a wearable device, an average heart rate at rest of a patient and a respiration rate of the patient; and
comparing the average heart rate at rest with a first threshold, wherein, if the average heart rate at rest is less than the first threshold, the patient is rejected.

2. The method of claim 1, further comprising:
comparing the average heart rate at rest with a second threshold, wherein, if the average heart rate at rest is greater than or equal to the first threshold and less than or equal to the second threshold, the patient is selected for an atropine test.

3. The method of claim 1, further comprising:
comparing the average heart rate at rest with the second threshold, wherein, if the average heart rate is greater than the second threshold, then a heart rate variability is calculated using at least one breathing cycle, wherein, if the heart rate variability is greater than or equal to a third threshold, the patient is rejected, and wherein, if the heart rate variability is less than the third threshold, the patient is suitable.

4. The method of claim 2, further comprising:
performing the atropine test on the patient selected for the atropine test by administering atropine and marking a start of the atropine test via the wearable device;
detecting and storing via the wearable device a change in the average heart rate at rest after the start of the atropine test; and
comparing the change in the average heart rate at rest with a fourth threshold, wherein, if the change is greater than the fourth threshold, the patient is rejected, and wherein, if the change is less than or equal to the fourth threshold, the patient is suitable.

5. The method of claim 4, wherein the start of the atropine test is marked by pushing a button on the wearable device.

6. The method of claim 4, wherein the change in the average heart rate at rest is calculated as the difference between a pre-test average heart rate and a post-test average heart rate, wherein the post-test average heart rate is calculated from a first period delayed from the start of the atropine test by a second period.

7. The method of claim 3, wherein the heart rate variability is calculated by:
analyzing an impedance signal representing respiration to detect an inspiration peak and an expiration peak;
searching for a peak heart rate following the inspiration peak and storing the peak heart rate;
searching for a minimum heart rate following the expiration peak and storing the minimum heart rate; and
calculating at least two differences between the peak heart rate and the minimum heart rate for at least two breathing cycles including the inspiration peak and the expiration peak, wherein the at least two differences are averaged as the heart rate variability.

8. The method of claim 7, wherein the signal representing respiration is an impedance signal.

9. A method for detecting heart rate according to respiration, the method comprising:
detecting and storing, via a wearable device, R-R intervals of a patient for a first time period;
detecting and storing, via the wearable device, respiratory intervals of the patient for the first time period;
detecting and storing, via the wearable device, accelerometer data of the patient for the first time period;
analyzing the accelerometer data and determining if motion is present in the first time period, wherein if motion is present, the R-R intervals and the respiratory intervals are deleted and detection is restarted;
calculating, if motion is not present in the first time period, an average R-R interval for the first time period by averaging all R-R intervals from the first time period;
storing the average R-R interval and respiratory intervals of the first time period; and
restarting detection of accelerometer data, the R-R intervals and respiratory intervals for a subsequent time period.

10. The method of claim 9, further comprising:
Transmitting, if a predetermined number of time periods have elapsed, for further processing stored average R-R intervals and respiratory intervals from the predetermined number of time periods to an electronic device for further analysis; and
averaging the stored average R-R intervals over the predetermined number of time periods to generate an extended average.

11. The method of claim 9, further comprising:
processing the R-R intervals and the respiratory intervals such that a heart rate variability is calculated, wherein calculation of the heart rate variability comprises:
detecting, for each respiratory interval, inspiration peaks and expiration peaks;
searching for a peak heart rate following each inspiration peak and storing the peak heart rate;
searching for a minimum heart rate following the expiration peak and storing the minimum heart rate; and
calculating at least two differences between the peak heart rate and the minimum heart rate over at least two breathing cycles in the inspiration interval, including the inspiration peak and the expiration peak, wherein the at least two differences are averaged as the heart rate variability for the respiratory interval;
storing the heart rate variability calculated for each respiratory interval; and
averaging the calculated heart rate variability of all stored respiratory intervals to generate an extended heart rate variability.

12. The method of claim 9, further comprising:
analyzing the accelerometer data to determine if the patient is in a supine position; and
identifying, if the patient is in a supine position, the detected R-R intervals and respiratory intervals as nighttime R-R intervals and nighttime respiratory intervals.

13. A monitoring system for a patient, comprising:
a wearable device including electrodes, a first processor, a computer-readable memory, an accelerometer, and an antenna; and
a mobile electronic device including a transceiver, a second processor and a display screen,
wherein the wearable device detects R-R intervals of heart rate and respiratory intervals of breathing via the electrodes,
wherein the wearable device stores the R-R intervals and respiratory intervals,
wherein the first processor processes the stored R-R intervals and respiratory intervals and transmits a processed heart rate and a processed respiratory interval to the mobile electronic device, and
wherein the mobile device analyzes the processed heart rate and processed respiratory intervals to determine if the patient is suitable for neuromodulation therapy and displays a determination result on the display screen.

14. The monitoring system of claim 13, wherein the wearable device further comprises:
a button for signaling a beginning of a clinical test.

15. The monitoring system of claim 14, wherein the clinical test is an atropine test.

16. The monitoring system of claim 13, wherein the wearable device is embedded in an adhesive patch for application to the patient.

17. The monitoring system of claim 13, wherein the respiratory intervals are detected from an impedance signal, and wherein the impedance signal is analyzed to identify points where a derivative is equal to zero to mark an inspiration peak or an expiration peak.

18. The monitoring system of claim 13, wherein the wearable device is connected to a desktop computer or a hospital server.

19. A method for assessing a suitability of neuromodulation therapy for a patient, the method comprising:
evaluating of at least two cardiac variables resulting in evaluation outcomes;
comparing the evaluation outcomes to threshold values; and
traversing a decision process for determining if a patient is a candidate for therapy, based on the comparison of the evaluation outcomes to the threshold values.
